(19) United States
(12) Patent Application Publication
(10) Pub. No.: US 2011/0236458 A1
(43) Pub. Date: Sep. 29, 2011

(54) COATING

(76) Inventors: David Franklin Farrar, York (GB); Kadem Gayad Al-Lamee, Leeds (GB); Jefferey Nmi Motley, Sheffield (GB)

(21) Appl. No.: 12/672,533

(22) PCT Filed: Aug. 7, 2008
(86) PCT No.: PCT/GB2008/002677

§ 371 Date: Jun. 10, 2011

(30) Foreign Application Priority Data
Aug. 7, 2007 (GB) 0715376.0

(51) Int. Cl. A61F 2/00; A61K 47/34

(52) U.S. Cl. 424/426; 514/772.3; 427/2.24

(57) ABSTRACT

The invention relates to a coating composition for an implantable medical device, a method of coating a medical device and a device coated with the composition.

### FIELD OF THE INVENTION

The invention relates to a coating composition for an implantable medical device, a method of coating a medical device and a device coated with the composition.

### BACKGROUND TO THE INVENTION

Stents are small expandable metal tubes that are implanted in arteries to keep them open in patients whose vessels have become blocked due to coronary artery disease, the most common cause of death in the Western World. Bare metal stents sometimes become blocked again (restenosis) requiring a re-intervention procedure to re-open them.

A drug-eluting stent, sometimes referred to as a “coated” or “medicated” stent, is a normal metal stent that has been coated with a pharmacologic (drug) that is known to interfere with the process of restenosis (re-narrowing). Restenosis has a number of causes; it is a very complex process and the solution to its prevention is equally complex. However, in the data gathered so far, the drug-eluting stent has been extremely successful in reducing restenosis from the 20-30% range to single digits.

After stent implantation, in addition to aspirin, the patient must take an anti-clotting or anti-platelet drug, such as clopidogrel or ticlopidine (brand names Plavix and Ticlid) for six or more months after stenting, to prevent the blood from reacting to the new device by thickening and clogging up the newly expanded artery (thrombosis). Ideally a smooth, thin layer of endothelial cells (the inner lining of the blood vessel) grows over the stent during this period and the device is incorporated into the artery, reducing the tendency for clotting.

Despite the clinical and commercial success of drug-eluting stents, recent evidence has suggested that they result in higher rates of late stage thrombosis, a clot formation at the end of the stent, which, in over 50% of cases, results in either an acute heart attack or death. Many physicians believe that the clot formations are due to the delayed healing of the vessel caused by the cytotoxic drugs that coat the stent or by some polymers that are used to deliver the drug. In response to these concerns some physicians are defaulting to using bare metal stents, where late stent thrombosis is not seen as much of a problem.

Most of the bioresorbable coatings used for the drug-eluting stents are based on poly(lactide), poly(glycolide) or co-polymers of the two (poly(lactide-co-glycolide)). The drug elution profile is controlled largely by control of the hydrophilicity/hydrophobicity of the polymer (typically determined by the lactide:glycolide ratio). Unfortunately the same balance of hydrophilicity/hydrophobicity also controls degradation rate. This means that in the current polymer coatings the drug elution profile is intrinsically bound up with degradation profile. Hence each new drug may need extensive reformulation of the polymer to obtain the correct elution profile. This could lead to lengthy development and regulatory timescales.

Another problem with coatings on stents is the adhesion of the polymer to the metal surface. Most polymer coatings will not adhere strongly to a normal metal surface and the coating may delaminate when the stent is deployed inside the vessel.

As a result, there is a requirement for a new generation of drug-eluting stents which have bioresorbable coatings in place of the current stable (non-resorbable) coatings. In this way the stent can deliver its payload of a drug from the coating but once the drug has been delivered the coating resorbs so that the stent effectively reverts to a bare metal stent, with lower chance of late stent thrombosis.

### SUMMARY OF THE INVENTION

According to an aspect of the invention there is provided a bioresorbable coating composition for an implantable medical device, the composition comprising a polymer and at least one additive which is an acid or a derivative thereof selected from the group consisting of hexanoic acid, octanoic acid, decanoic acid, lauric acid, myristic acid, crotonic acid, 4-pentenoic acid, 2-hexenoic acid, undecylenic acid, petroselenic acid, oleic acid, erucic acid, 2,4-hexadienoic acid, linoleic acid, linolenic acid, benzoic acid, hydrocinnamic acid, 4-isopropylbenzoic acid, ibuprofen, ricinoleic acid, adipic acid, suberic acid, phthalic acid, 2-bromolauric acid, 2,4-hydroxydodecanoic acid, butyric acid, monobutyrin, 2-hexyldecanoic acid, 2-butyloctanoic acid, 2-ethylhexanoic acid, 2-methylvaleric acid, 3-methylvaleric acid, 4-methylvaleric acid, 2-ethylbutyric acid, trans-beta-hydromuconic acid, isovaleric anhydride, hexanoic anhydride, decanoic anhydride, lauric anhydride, myristic anhydride, 4-pentenoic anhydride, oleic anhydride, linoleic anhydride, benzoic anhydride, poly(azelaic anhydride), 2-octen-1-yl succinic anhydride and phthalic anhydride and wherein the composition further comprises at least one drug.

The coating composition of the invention is a drug-eluting bioresorbable coating. The use of the additive at least partially decouples the relationship between the degradation and elution profiles of a particular polymeric species and allows control of the degradation and elution profiles.

We have found that it is possible to control the rate of degradation of polymers, in particular lactic acid polymers, by homogenously blending certain additives which are both fully miscible with the polymer and will not leach out. The blending process is simple and results in stable polymer blends which can be used as coatings on implantable medical devices, whereby the coatings will both maintain their physical strength yet biodegrade in a predictable manner.

In embodiments of the invention the polymer in the polymer blend is selected from the group consisting of a polyester, poly(trimethylene carbonate), polydioxanone, polyalkenoate, polyhydroxybutyrate, polyorthoester and any suitable copolymers or blends thereof.

Examples of suitable polyesters include poly α-hydroxy acids such as poly(lactic acid) and poly(glycolide). A further example of a suitable polyester is poly(caprolactone).

In further embodiments of the invention lactic acid polymer may be present as a homopolymer, for example a homopolymer of poly(L-lactide) (PLLA), poly(D,L-lactide) (PDLLA) or as a co-polymer, for example as poly(L-lactide-co-glycolide (PLLA co GA) and poly(D,L-lactide-co-glycolide) (PDLLA co GA).

In embodiments of the invention the lactic acid polymer or co-polymer is polymerised with caprolactone. Specifically the co-polymer is poly(D,L-lactide-co-glycolide-co-caprolactone).

For example, PLLA is a very hydrophobic polymer that has a slow drug release profile and a long degradation time. The incorporation of an additive into the polymeric coating composition accelerates the rate of degradation and modifies the drug elution profile.

The composition may also contain other polymeric components blended therewith.

The additive concentration is chosen such that it must be fully miscible with the polymer and should not leach out of the polymer.

As used herein the term “fully miscible” means that when a 0.5 mm thick sheet of the polymer is visually inspected the sheet is either uniformly transparent or, if the sheet is opaque, the opacity is uniform.

As used herein the term “not leach out of the polymer” is defined such that when a thin (thickness <1 mm) sample is immersed in an excess of PBS (phosphate buffered saline solution), at least half of the added additive remains in the sample after 1 week.

Aptly the composition contains the additive in an amount which is not more than 10%, typically not more than 5%, and even more typically not more than 2% by weight of the composition.

The amount of the additive chosen will also depend upon the rate of degradation desired. In vivo degradation occurs firstly by hydrolytic scission of the polymer chains resulting in the formation of units of increasingly smaller molecular weight until only substantially monomers remain. Thereafter, the monomers are metabolized and absorbed into the body. It is only in the last stages of degradation that mass loss occurs.

We have found that a preferred additive for use in the invention is lauric acid. This may be employed as the acid per se or, if desired, as a derivative, for example as the anhydride.

Preferred compositions will contain lauric acid a derivative thereof in an amount not more than 10%, more typically not more than 5%, and even more typically not more than 2% by weight of the composition.

The varying degradation rates of a composition comprising polylactic acid as the polymer component, wherein an additive is employed at 2% by weight of the polymer component are shown in the following table:

| Additive | Degradation rate (days⁻¹) |
|---|---|
| Hexanoic acid | −0.0565 |
| Octanoic acid | −0.0448 |
| Decanoic acid | −0.0472 |
| Lauric acid | −0.0326 |
| Myristic acid | −0.0281 |
| Crotonic acid | −0.0489 |
| 4-Pentenoic acid | −0.0567 |
| 2-Hexenoic acid | −0.0713 |
| Undecylenic acid | −0.07 |
| Petroselenic acid | −0.0542 |
| Oleic acid | −0.0442 |
| Erucic acid | −0.0315 |
| 2,4-Hexadienoic acid | −0.0618 |
| Linoleic acid | −0.0488 |
| Linolenic acid | −0.0589 |
| Benzoic acid | −0.0798 |
| Hydrocinnamic acid | −0.0737 |
| 4-Isopropylbenzoic acid | −0.0728 |
| Ibuprofen | −0.051 |
| Ricinoleic acid | −0.061 |
| Adipic acid | −0.0373 |
| Suberic acid | −0.0311 |
| Phthalic acid | −0.0855 |
| 2-Bromolauric acid | −0.0769 |
| 2,4-Hydroxydodecanoic acid | −0.0318 |
| Monobutyrin | −0.0347 |
| 2-Hexyldecanoic acid | −0.0339 |
| 2-Butyloctanoic acid | −0.0467 |
| 2-Ethylhexanoic acid | −0.0473 |
| 2-Methylvaleric acid | −0.0411 |
| 3-Methylvaleric acid | −0.0587 |
| 4-Methylvaleric acid | −0.0553 |
| 2-Ethylbutyric acid | −0.053 |
| Trans-beta-hydromuconic | −0.039 |
| acid | |
| Isovaleric anhydride | −0.0628 |
| Hexanoic anhydride | −0.0919 |
| Decanoic anhydride | −0.0807 |
| Lauric anhydride | −0.0698 |
| Myristic anhydride | −0.0626 |
| 4-Pentenoic anhydride | −0.0888 |
| Oleic anhydride | −0.0504 |
| Linoleic anhydride | −0.0696 |
| Benzoic anhydride | −0.0817 |
| Poly(azelaic anhydride) | −0.0784 |
| 2-Octen-1-yl succinic | −0.1012 |
| anhydride | |
| Phthalic anhydride | −0.0841 |

In a specific embodiment of the invention the polymeric component is PLLA and the additive is lauric acid or derivatives thereof in amounts of more than 10%, not more than 5% and typically not more than 2% by weight of the polymer component.

A further embodiment of the present invention provides the provision of an additive which not only will control the rate of degradation but will delay the onset of the additive-induced degradation process. This delay may be achieved, aptly by the use of additives which are convertible to the acidic form of the additive. Suitable derivatives are acid anhydrides which will, in an in vivo environment hydrolyse to the corresponding acid. Preferred anhydrides include lauric anhydride and benzoic anhydride, in amounts of, aptly, not more than 5%, more aptly, not more than 2% and, typically, not more than 1% by weight of the polymer blend.

A “drug” is herein defined as any chemical substance used in the treatment, cure, prevention, or diagnosis of disease or used to otherwise enhance physical or mental well-being. Examples of a suitable drug type for incorporation into the coating composition of the present invention include, an anti-inflammatory agent, a cytotoxic agent, an angiogenic agent, an osteogenic agent, an immunosuppressant, an anti-clotting agent, an anti-platelet agent, an antimicrobial or an antibiotic.

Suitable anti-platelet agents include clopidogrel, sold as Plavix™ by Bristol Myers-Squibb and ticlopidine, sold as Ticlid™ by Sanofi-Aventis.

Suitable immunosuppressants include rapamycin, also known as Sirolimus available from A.G Scientific Inc.

Suitable antibiotics include gentamicin and vancamycin.

In embodiments of the invention the additive is also the drug. For example, monobutyrin can be used as the additive to modify the degradation rate of the polymer, whilst its inherent angiogenic and osteogenic properties can also be used to treat a subject.

According to a further aspect of the invention there is provided an implantable medical device having a first coating of a composition according to the invention. This first coating composition can be applied directly to the device. Alternatively the first coating composition is indirectly applied to the surface of the device. This indirect application of the first coating composition to the medical device can be via the functionalisation of at least part of the surface to provide suitable functional groups for bonding to a polymer. Functionalisation can, for instance, result in the first coating composition being covalently bound/coupled to the surface of the device.

Most polymer coatings will not adhere strongly to a normal metal surface and the coating has a tendency to de-laminate. Functionalisation improves the adhesion of the polymeric coating to the surface of the medical device and minimises the risk of delamination.

In order to improve the adhesion of a polymer coating to the metal surface, a chemical bridge is used to link the two together. In the first stage of the functionalisation process a chemical is chosen that reacts well with the inherent functionality of the metal surface. It reacts with the oxides, hydroxides, epoxide or any other surface oxide on metallic surfaces to form strong bonds whilst leaving the rest of the molecule free to react with other species.

Suitable chemicals for use in the first reaction step include alkoxysilanes of the formula (RO)₃Si(R¹X) wherein R represents methyl or ethyl and R¹ represents C₂-C₁₀ alkyl in which one or more methylene groups may be replaced by —NH— or —O—, C₂-C₁₀ cycloalkyl or cycloalkylalkyl, C₂-C₁₀ aralkyl or monocylic or bicyclic aryl and X represents amino, hydroxyl, carboxylic acid or acid anhydride. Preferably R¹ represents C₂-C₁₀ alkyl in which one or more methylene groups is optionally replaced by —NH— and X represents —NH₂, and an example of a suitable priming agent is N-[3-(trimethoxysilyl)propyl]ethylenediamine.

In the second stage, another chemical is chosen which reacts readily with the functional/reactive groups of the first chemical and which also has a functional group which can react with oxygen containing groups in the polymer, for example, hydroxyl, methoxy and ethoxy groups. A strong bond is therefore formed between the two molecules and the polymer is coupled to the functionalised surface. In this way a strong chemical bond is achieved between the functionalised surface and the polymer, improving the adhesion of the polymer to the metal surface.

Any chemical with an alkoxysilyl group on one end and an isocyanate on the other end is suitable for use in the second reaction step. An example of an appropriate chemical is 3-(triethoxysilyl)propylisocyanate.

FIGS. 1 and 2 illustrate the functionalisation process.

In further embodiments of the invention a second coating composition is provided between at least part of the surface of the device and the first coating. This second coating composition is referred to as a “tie-coat”. This composition comprises a low molecular weight polymer of similar or identical chemical composition to the polymer component of the first coating composition. The polymer of the second coating composition can be any polymer which is capable of forming a strong adhesion with the polymer component of the first coating composition.

In embodiments of the invention the first and second coating compositions contain —OH groups to react with the triethoxy groups from the second functionalisation step.

For example the polymeric component of the first coating can be PLLA (MW 125k) and the polymeric component of the second coating can be PDLLA co GA (MW 5-15k).

The second coating composition can be applied directly to the surface of the device and then the first coating composition applied to the second coating composition. Alternatively, the surface of the device can be functionalised as described above, the second coating composition thus being covalently coupled via the functional molecules to the surface of the device and then the first coating composition being applied to the second coating composition.

The second coating composition can further comprise at least one additive which is an acid or a derivative thereof selected from the group consisting of hexanoic acid, octanoic acid, decanoic acid, lauric acid, myristic acid, crotonic acid, 4-pentenoic acid, 2-hexenoic acid, undecylenic acid, petroselenic acid, oleic acid, erucic acid, 2,4-hexadienoic acid, linoleic acid, linolenic acid, benzoic acid, hydrocinnamic acid, 4-isopropylbenzoic acid, ibuprofen, ricinoleic acid, adipic acid, suberic acid, phthalic acid, 2-bromolauric acid, 2,4-hydroxydodecanoic acid, butyric acid, monobutyrin, 2-hexyldecanoic acid, 2-butyloctanoic acid, 2-ethylhexanoic acid, 2-methylvaleric acid, 3-methylvaleric acid, 4-methylvaleric acid, 2-ethylbutyric acid, trans-beta-hydromuconic acid, isovaleric anhydride, hexanoic anhydride, decanoic anhydride, lauric anhydride, myristic anhydride, 4-pentenoic anhydride, oleic anhydride, linoleic anhydride, benzoic anhydride, poly(azelaic anhydride), 2-octen-1-yl succinic anhydride and phthalic anhydride.

The second coating composition can further comprise at least one drug. Examples of a suitable drug include, but are not limited to, an anti-inflammatory agent, a cytotoxic agent, an angiogenic agent, an osteogenic agent, an immunosuppressant, an anti-clotting agent, an anti-platelet agent, an antimicrobial or an antibiotic.

Medical devices for which this coating technology may be advantageous, include stents, orthopaedic implants, dental implants and maxillo-facial implants.

Examples of stents to which the drug-eluting bioresorbable coating of the invention can be applied include coronary stents, for example carotid stents, aortic stents, renal stents and venous stents. Other examples of stents include peripheral stents. Examples of orthopaedic implants to which the drug-eluting bioresorbable coating can be applied include reconstructive and trauma products, for example, components of hip replacement, components of knee replacements, fracture plates, screws, pins, external fixation plates, intramedullary nails, interference screws, suture anchors.

Examples of maxillo-facial implants to which the drug-eluting bioresorbable coating of the invention can be applied include plates, screws and meshes.

According to a further aspect of the invention there is provided a method of coating a medical device comprising the step of:
a) applying to at least part of the device the first coating composition according to the present invention.

The method can additionally comprise the step of:
b) applying a second coating composition to at least part of the device, wherein the second coating composition is applied prior to the first coating composition, and wherein the second coating composition has a polymeric component having a lower molecular weight than that of the first coating composition.

In further embodiments of the invention at least part of the surface of the medical device is functionalised prior to the application of the first coating composition or the second coating composition.

The process of functionalisation is outlined in detail above with reference to FIGS. 1 and 2.

According to a further aspect of the invention there is provided a method of using a device which is coated with a drug-eluting bioresorbable coating of the invention, wherein the method comprises the step of implanting the device in an animal or human body.

The implantable medical device can be, for example a stent, an orthopaedic implant or a dental implant. Examples of suitable stents include coronary stents, for example carotid stents, aortic stents, renal stents and venous stents. Other examples of stents include peripheral stents.

According to a still further aspect of the invention there is provided a vehicle for carrying a drug, wherein the vehicle is defined as the composition of the present invention.

Further areas of applicability of the present invention will become apparent from the detailed description provided hereafter. It should be understood that the detailed description and specific examples, while indicating the preferred embodiment of the invention, are intended for purpose of illustration only and are not intended to limit the scope of the invention.

### BRIEF DESCRIPTION OF THE DRAWINGS

The accompanying drawings, which are incorporated in and form part of the specification, illustrate the embodiments of the present invention and together with the written description serve to explain the principles, characteristics, and features of the invention. In the drawings:

FIG. 1: Illustrative chemistry

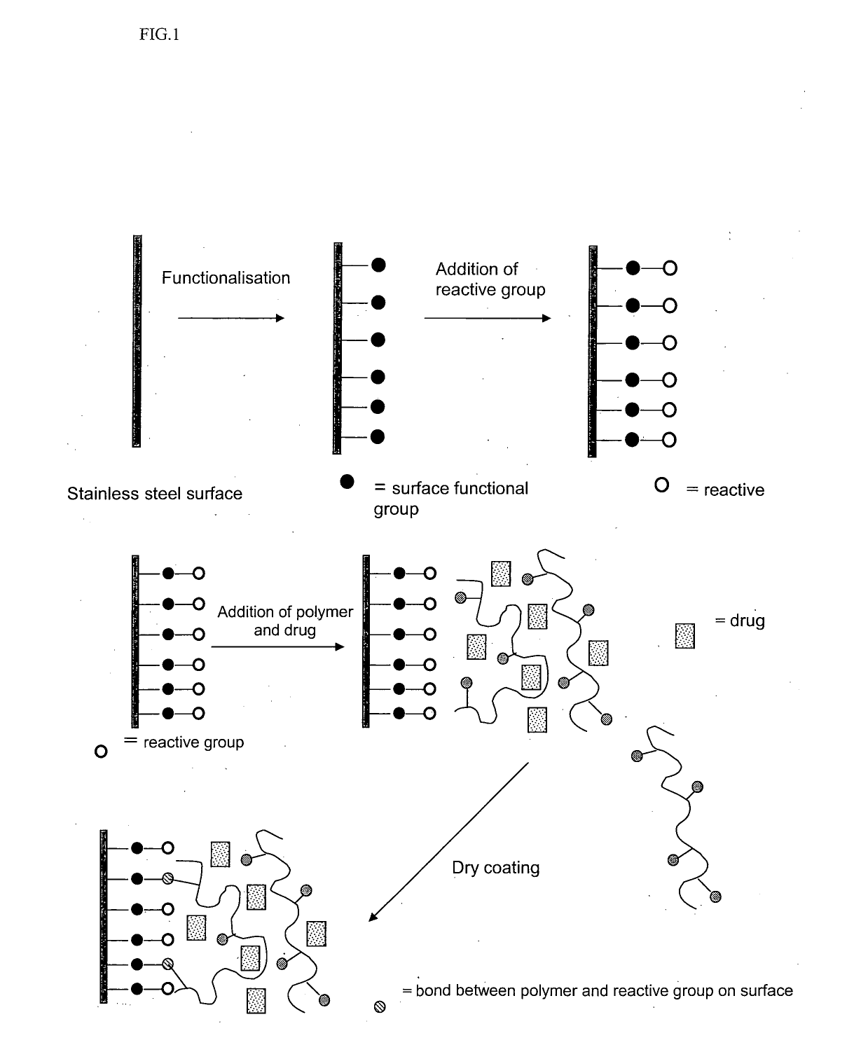

FIG. 2: Schematic of surface functionalisation

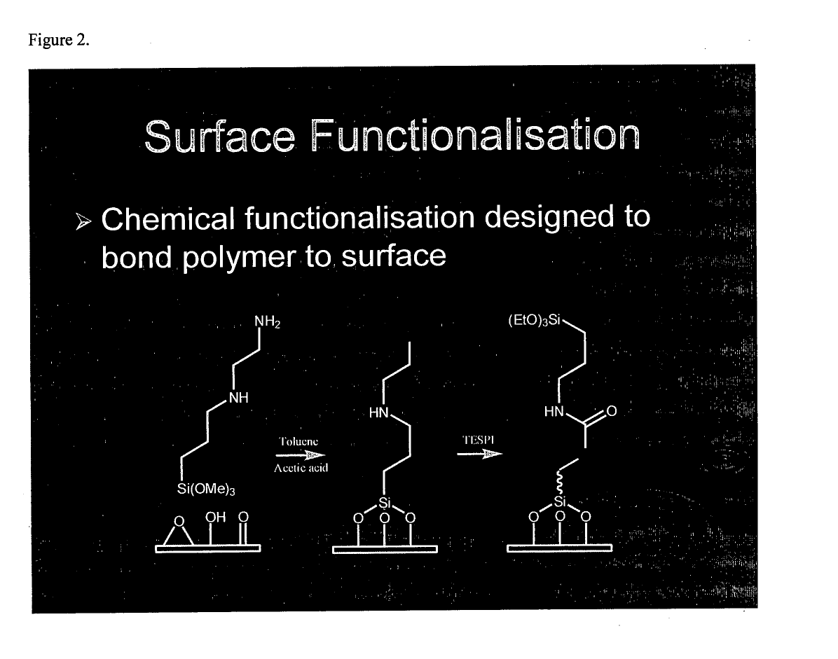

FIG. 3: Elution of rapamycin into HBS-EP buffer from PLLA

FIG. 4: Elution of rapamycin into PBS buffer from PLGA

FIG. 5: In-vitro degradation testing of coatings

### DETAILED DESCRIPTION OF THE INVENTION

### Materials

Stainless steel 316L: Goodfellow
Sodium hydrogen carbonate: Sigma Aldrich
N-[3-(trimethoxysilyl)propyl]ethylenediamine (TMSPEA): Sigma Aldrich
Glacial acetic acid: Sigma Aldrich
3-(triethoxysilyl)propyl isocyanate (TESPI): Sigma Aldrich

### Poly(D,L-lactide-co-glycolide) (PLGA1) 50:50, Mw 5-15k: Sigma Aldrich

### Poly(L-lactide) (PLLA), Mw 125k: Smith and Nephew

### Poly(D,L-lactide-co-glycolide) (PLGA2) 50:50, Mw 10k: Durect

### Poly(D,L-lactide-co-glycolide) (PLGA3) 50:50, Mw 30k: Durect

### Poly(D,L-lactide-co-glycolide-co-caprolactone) (PLGC1) 47.5:40:12.5: Smith and Nephew

Lauric acid (LA): Sigma Aldrich
Sodium dodecyl sulphate (SDS): Sigma Aldrich
Rapamycin: LC laboratories

### HBS-EP Buffer: GE Healthcare

### Phosphate Buffered Saline (PBS): Sigma Aldrich

### Solvents: Sigma Aldrich

### Example 1

### Stage 1

### Cleaning

Stainless steel samples (50 mm×17 mm×0.1 mm annealed finish) were cleaned by sonication for 15 minutes in a 7.5% w/w solution of aqueous sodium hydrogen carbonate, rinsing in deionised water, sonication for 15 minutes in 2-propanol and sonication for 15 minutes in deionised water. The samples were then dried at 100° C. for 16 hours, followed by drying at 50° C. for 30 minutes.

### Stage 2

### Functionalisation

2.5 ml glacial acetic acid solution in toluene (1.0% w/w, 0.4 mmol) was added to 200 ml toluene followed by 1.6 ml TMSPEA and mixed. The samples were removed from the 50° C. oven and immersed in the solution for 5 minutes. The samples were removed and kept at 50° C. for 20 hours.

The samples were rinsed in a series of solvents by rotating sequentially for 15 minutes in each of toluene, methanol, deionised water, methanol and deionised water. Finally, the samples were rinsed for 5 minutes in methanol and then dried at 50° C. for 2 hours.

Anhydrous toluene was added, under nitrogen, into a measuring cylinder being purged with nitrogen. Enough TESPI was added to give a 4% v/v solution in toluene. The samples (dried at 50° C. for 15 minutes and allowed to cool for two minutes before use) were immersed in the solution on a holder and rotated under nitrogen for 15 minutes. The samples were then rinsed in anhydrous toluene under nitrogen and dried under vacuum for 16 hours.

### Stage 3

### Applying a “Tie-Coat”

The samples were placed on a hotplate at approximately 50° C. and primed with 1% w/w PLLA or PLGA1 solutions in CHCl₃. The priming was performed using a handheld spray gun working at 10 psi from a distance of approximately 15 cm. Between 2 and 4 passes were needed, depending on the speed of movement, to achieve a primer coat weight of between 50 and 100 μg per cm². The samples were then cured for 16 hours at 100° C.

### Stage 4

### Coating

After curing at 100° C. the samples were cooled for 5 minutes and then placed on a hotplate at approximately 50° C. and coated with a 1% w/w PLLA solution in CHCl₃. The coating was performed using a handheld spray gun working at 10 psi from a distance of approximately 15 cm. Between 20 and 40 passes were needed, depending on the speed of movement, to achieve a coat weight of 600-700 μg per cm². The samples were then dried under vacuum at 50° C. for 16 hours.

### Stage 5

### Testing

The adhesion tests were performed using a ‘finger-rub’ shear test and the results were placed into a category as shown below.

| Testing Category | Explanation |
|---|---|
| A | Coating is removed easily with finger rubbing |
| B | Coating is removed after rubbing with fingernail |
| C | Coating is removed after vigorous rubbing with |
| | fingernail |
| D | Coating is not removed after vigorous rubbing with |
| | fingernail |

Prior to testing, all samples were scored with a scalpel 1 cm and 3 cm from the bottom of the plate and then immersed in a 0.02% w/w solution of SDS for 1 hour.

| Sample | Primer and Topcoat | Test results |
|---|---|---|
| 1 | PLGA1, PLLA | D |
| 2 | PLGA1, PLLA | D |
| 3 | PLLA, PLLA | A |
| 4 | PLLA, PLLA | B |
| 5 | PLLA, PLLA | A |

Samples 1-4 were functionalised as previously explained. Sample 5 was cleaned but left unfunctionalised. The use of a lower molecular weight poly(lactide) based polymer as a primer coat was designed to increase the number of OH end groups available for reaction with the surface functionalisation and to therefore improve the adhesion of the coating to the metal surface. The results show that the use of PLGA1 as a primer greatly increases the adhesion of the coating to the metal surface.

### Example 2

Stainless steel plates were functionalised and primed in an identical manner to Example 1. 1% w/w solutions in CHCl₃ of PLLA and lauric acid (99:1, 98:2, 96:4) were prepared. The coating and testing then followed the procedure outlined in Example 1.

| Sample | Primer and Topcoat | Test results |
|---|---|---|
| 1 | PLGA1, PLLA + 1% LA | D |
| 2 | PLGA1, PLLA + 2% LA | D |
| 3 | PLGA1, PLLA + 4% LA | C |
| 4 | PLGA1, PLLA + 1% LA | C |
| 5 | PLGA1, PLLA + 2% LA | C |
| 6 | PLGA1, PLLA + 4% LA | B |
| 7 | PLGA1, PLLA | D |
| 8 | PLGA1, PLLA | B |

Samples 1-7 were functionalised as previously explained. Sample 8 was left unfunctionalised.

The results show that functionalisation of the stainless steel surfaces improves adhesion of the coating to the metal surface. They also show that the addition of lauric acid decreases the amount of adhesion of the coating to the metal surface and that higher percentages of lauric acid cause a larger loss of adhesion.

### Example 3

A commercially available stainless steel stent was prepared as in Example 1, up to and including stage 2 and then coupled with PLGA as described below.

### PLGA Coupling—“Tie Coat”

The stents were attached to a mandrel and coated with a primer solution containing 0.5% w/w PLGA1 in CHCl₃ on a Sonotek MediCoat Benchtop Coater. The parameters used were: 0.075 ml/min flow rate, 0.8 W ultrasonic power, 4 passes, 40 rpm rotation, 0.13 cm/s horizontal travel and 25 mm from stent to spray head. After priming, the stents were left for 16 hours at 100° C.

### Coating

After cooling for 5 minutes, the stents were attached to a mandrel and coated on the Sonotek MediCoat Benchtop Coater with a 0.5% w/w solution in CHCl₃ of PLLA, rapamycin and lauric acid (75:25:0 and 74:25:1). The parameters used were: 0.075 ml/min flow rate, 0.8 W ultrasonic power, 20 passes, 40 rpm rotation, 0.13 cm/s horizontal travel and 25 mm from stent to spray head. After coating, the stents were dried under vacuum for 16 hours at 40° C.

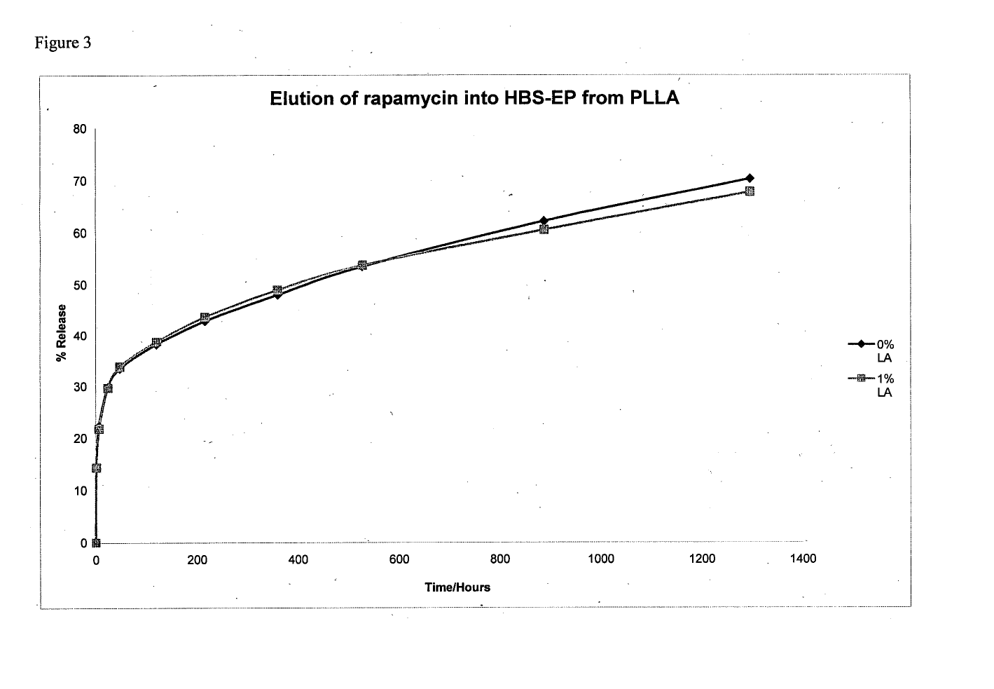

The stents were released into HBS-EP buffer (20 mM HEPES, 150 mM NaCl, 3 mM EDTA, pH 7.5) (Biacore™, GE Healthcare) at 37° C. and the elution monitored by UV/vis spectroscopy. Fresh buffer solution was added after each reading and the cumulative absorbance at 279 nm was recorded. The results, as illustrated in FIG. 3, show that rapamycin is released from a PLLA coating and that the addition of lauric acid has a negligible effect on the rate of drug elution.

### Example 4

A commercially available stainless steel stent was prepared as in Example 1, up to and including stage 1 and then primed with PLGA1 as described below:

### PLGA Coupling—“Tie Coat”

The stents were attached to a mandrel and coated with a primer solution containing 0.5% w/w PLGA1 in CHCl₃ on a Sonotek MediCoat Benchtop Coater. The parameters used were: 0.075 ml/min flow rate, 0.8 W ultrasonic power, 2 passes, 40 rpm rotation, 0.13 cm/s horizontal travel and 25 mm from stent to spray head. After priming, the stents were left for 16 hours at 100° C.

### Coating

After cooling for 5 minutes, the stents were attached to a mandrel and coated on the Sonotek MediCoat Benchtop Coater with a 0.5% w/w solution in CHCl₃ of PLGA2, PLGA3, rapamycin and lauric acid (30:45:25:0, 29.2:43.8:25:2 and 28.4:42.6:25:4). The parameters used were: 0.075 ml/min flow rate, 0.8 W ultrasonic power, 20 passes, 40 rpm rotation, 0.13 cm/s horizontal travel and 25 mm from stent to spray head. After coating, the stents were dried under vacuum for 16 hours at 40° C.

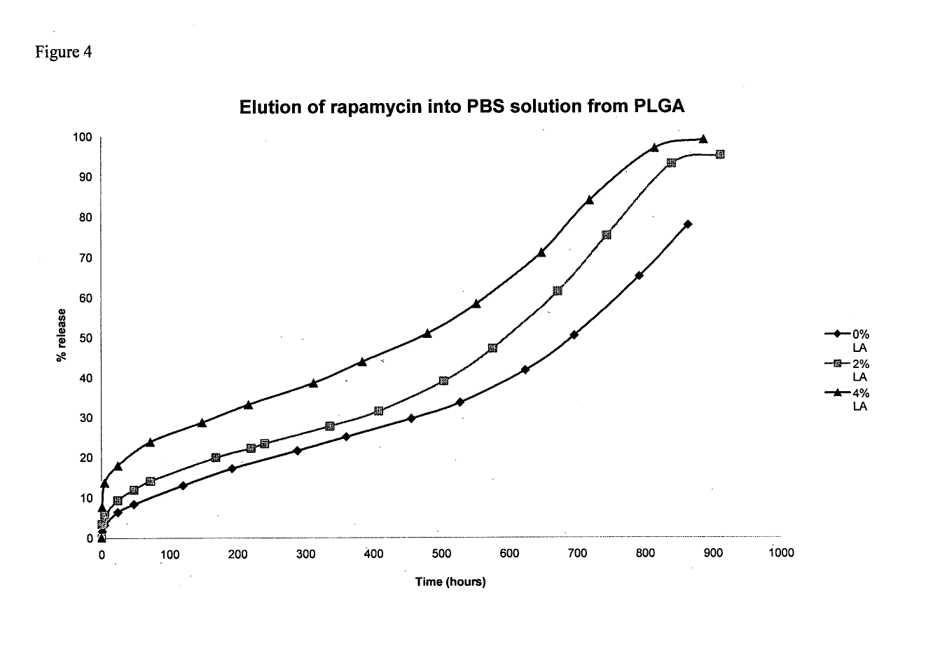

The stents were released into a 1% w/w PBS solution at 37° C. and the elution monitored by UV/vis spectroscopy. Fresh solution was added after each reading and the cumulative absorbance at 279 nm was recorded. The results, as illustrated in FIG. 4, show that rapamycin is released from a blended PLGA coating and that the addition of lauric acid has an effect on the initial burst of drug but less effect on the subsequent rate of drug elution.

### Example 5

Two 316L stainless steel plates (50 mm×50 mm×0.25 mm) were prepared as in Example 1, up to and including stage 1.

A polymer film was then cast on either plate using a 1% solution in CHCl₃ of PLGC1, rapamycin and lauric acid (80:20:0 and 78:20:2). Sufficient polymer was cast to achieve a film weight of approximately 100 mg over the plate. The films were dried under vacuum for 16 hours at 40° C.

After drying, each of the polymer-coated stainless steel plates was cut into nine coupons for in-vitro degradation testing.

### In-Vitro Degradation Testing

The coated coupons were immersed in phosphate buffered saline (PBS) solution at a pH of 7.4 and maintained in an incubator at a temperature of 37° C. Samples were removed at pre-determined time-points and the molecular weight of the coating polymer was measured using gel permeation chromatography (GPC).

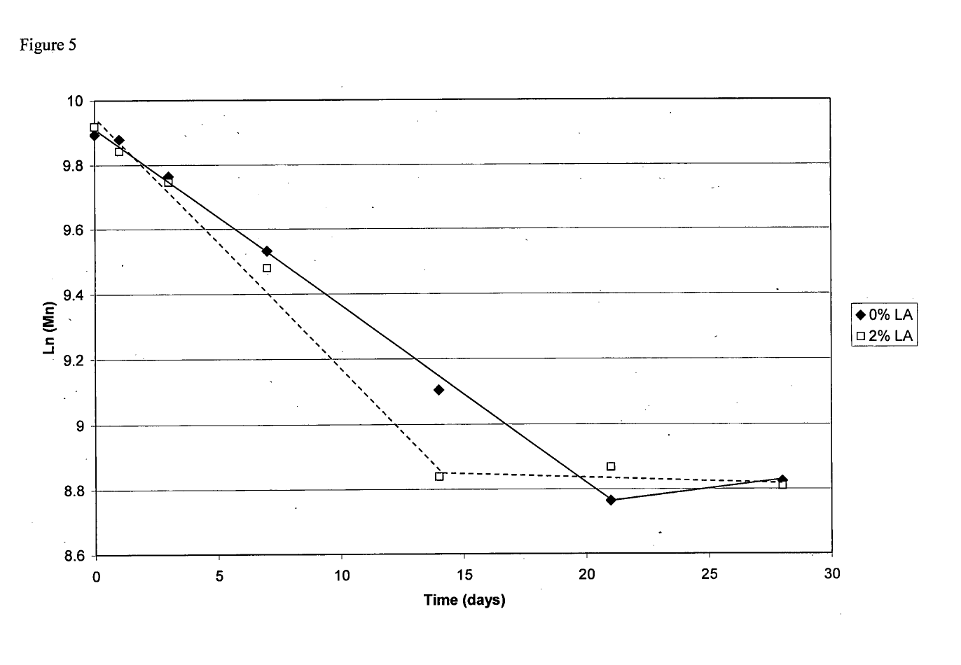

The results are shown in FIG. 5 where they are plotted as the natural logarithm of number average molecular weight i.e. ln(Mn) vs. time.

The results show that the plots are initially linear confirming a good fit to an “autocatalytic degradation model” where:

ln M(t)=ln M₀−kt

and M(t) is the molecular weight at time t, M₀ is the initial molecular weight at t=0 and k is the rate constant of degradation. The rate constant k is obtained from the gradient of the linear fit to the data.

The discontinuity in the linear plots indicated by a change in gradient is believed to be associated with the onset of significant mass loss and loss of low molecular weight soluble components.

From the initial slope of the graphs in the figure the following degradation rates were obtained:

0% lauric acid: k=0.0557 day⁻¹
2% lauric acid: k=0.0764 day⁻¹

These results show that the addition of 2% lauric acid to this coating increases the degradation rate by 37%.

The results also show that the discontinuity associated with onset of mass loss changes from around 21 days with 0% lauric acid to 14 days for 2% lauric acid further confirming the reduction in degradation time caused by the additive.

1. A bioresorbable coating composition for an implantable medical device, the composition comprising a polymer blend comprising at least one polymer in admixture with an amount of at least one additive comprising an acid or a derivative thereof selected from the group consisting of hexanoic acid, octanoic acid, decanoic acid, lauric acid, myristic acid, crotonic acid, 4-pentenoic acid, 2-hexenoic acid, undecylenic acid, petroselenic acid, oleic acid, erucic acid, 2,4-hexadienoic acid, linoleic acid, linolenic acid, benzoic acid, hydrocinnamic acid, 4-isopropylbenzoic acid, ibuprofen, ricinoleic acid, adipic acid, suberic acid, phthalic acid, 2-bromolauric acid, 2,4-hydroxydodecanoic acid, butyric acid, monobutyrin, 2-hexyldecanoic acid, 2-butyloctanoic acid, 2-ethylhexanoic acid, 2-methylvaleric acid, 3-methylvaleric acid, 4-methylvaleric acid, 2-ethylbutyric acid, trans-beta-hydromuconic acid, isovaleric anhydride, hexanoic anhydride, decanoic anhydride, lauric anhydride, myristic anhydride, 4-pentenoic anhydride, oleic anhydride, linoleic anhydride, benzoic anhydride, poly(azelaic anhydride), 2-octen-1-yl succinic anhydride and phthalic anhydride, wherein the polymer blend further comprises at least one drug.

2. The composition of claim 1, wherein the polymer is selected from the group consisting of a polyester, poly(trimethylene carbonate), polydioxanone, polyalkenoate, polyhydroxybutyrate, polyorthoester, copolymers thereof, or blends thereof.

3. The composition of claim 2, wherein the polymer comprises polyester and wherein the polyester is selected from the group consisting of polylactic acid, polyglycolide and polycaprolactone.

4. The composition of claim 2, wherein the polymer comprises polyester and wherein the polyester is a homopolymer or a co-polymer comprising poly(L-lactide) (PLLA), poly-(D,L-lactide) (PDLLA), poly(L-lactide-co-glycolide) (PLLA-co-GA) or poly(D,L-lactide-co-glycolide (PDLLA-co-GA).

5. The composition of claim 4, wherein the polyester comprises a co-polymer and wherein the co-polymer further comprises caprolactone.

6. The composition of claim 5, wherein the co-polymer comprises poly(D,L-lactide-co-glycolide-co-caprolactone).

7. The composition of claim 1, wherein the amount of the additive is not more than 10% by weight of the composition.

8. The composition of claim 7, wherein the amount of the additive is not more than 5% by weight of the composition.

9. The composition of claim 8, wherein the amount of the additive is not more than 2% by weight of the composition.

10. The composition of claim 4, wherein the polyester comprises PLLA and wherein the additive comprises lauric acid or a derivative thereof.

11. The composition of claim 10, wherein the composition comprises not more than 10% by weight of lauric acid or a derivative thereof.

12. The composition of claim 11, wherein the composition comprises not more than 5% by weight of lauric acid or a derivative thereof.

13. The composition of claim 1, wherein the drug is selected from the group consisting of an anti-inflammatory agent, a cytotoxic agent, an angiogenic agent, an osteogenic agent, an immunosuppressant, an anti-clotting agent, an anti-platelet agent, an antimicrobial and an antibiotic.

14. An implantable medical device having a first coating composition comprising the composition of claim 1.

15. The medical device of claim 14, wherein the first coating composition is applied directly to a surface of the device.

16. The medical device of claim 14, wherein the first coating composition is indirectly applied to a surface of the device.

17. The medical device of claim 14, wherein the first coating composition is applied to the device and wherein at least part of a surface of the device is functionalised such that the first coating composition is covalently coupled to the device via a functional molecule.

18. The medical device of claim 17, wherein the functional molecule comprises an alkoxysilane group which reacts with oxides or hydroxides on the surface of the device to form covalent bonds and wherein the polymer of the first coating composition is covalently coupled to the surface via the functional molecule.

19. The medical device of claim 18, wherein the functional molecule further comprises one or more amino, hydroxyl, carboxylic acid or acid anhydride groups.

20. The medical device of claim 14, wherein the medical device further comprises a second coating composition provided between at least part of a surface of the device and the first coating composition.

21. The medical device of claim 20, wherein the polymer of the first coating composition has a molecular weight and wherein the second coating composition comprises a polymer having a lower molecular weight than the molecular weight of the polymer of the first coating composition.

22. The medical device of claim 21, wherein the polymer of the first coating composition comprises PDLLA and the polymer of the second coating composition comprises PLA-co-GA.

23. The medical device of claim 20, wherein the second coating composition comprises at least one additive which is an acid or a derivative thereof selected from the group consisting of hexanoic acid, octanoic acid, decanoic acid, lauric acid, myristic acid, crotonic acid, 4-pentenoic acid, 2-hexenoic acid, undecylenic acid, petroselenic acid, oleic acid, erucic acid, 2,4-hexadienoic acid, linoleic acid, linolenic acid, benzoic acid, hydrocinnamic acid, 4-isopropylbenzoic acid, ibuprofen, ricinoleic acid, adipic acid, suberic acid, phthalic acid, 2-bromolauric acid, 2,4-hydroxydodecanoic acid, butyric acid, monobutyrin, 2-hexyldecanoic acid, 2-butyloctanoic acid, 2-ethylhexanoic acid, 2-methylvaleric acid, 3-methylvaleric acid, 4-methylvaleric acid, 2-ethylbutyric acid, trans-beta-hydromuconic acid, isovaleric anhydride, hexanoic anhydride, decanoic anhydride, lauric anhydride, myristic anhydride, 4-pentenoic anhydride, oleic anhydride, linoleic anhydride, benzoic anhydride, poly(azelaic anhydride), 2-octen-1-yl succinic anhydride and phthalic anhydride.

24. The medical device of claim 20, wherein the second coating composition comprises at least one drug.

25. The medical device of claim 24, wherein the drug is selected from the group consisting of an anti-inflammatory agent, a cytotoxic agent, an angiogenic agent, an osteogenic agent, an immunosuppressant, an anti-clotting agent, an anti-platelet agent, an antimicrobial and an antibiotic.

26. The medical device of claim 14, wherein the medical device comprises a stent, an orthopaedic implant or a dental implant.

27. The medical device of claim 26, wherein the medical device comprises a stent and wherein the stent comprises a coronary stent, a carotid stent, an aortic stent, a renal stent, a venous stent or a peripheral stent.

28. A method of coating a medical device comprising a surface, the method comprising:
applying a first coating composition comprising the coating composition of claim 1 to the device.

29. The method of claim 28, further comprising functionalizing at least part of the surface prior to applying the first coating composition.

30. The method of claim 28, further comprising
applying a second coating composition to the device prior to applying the first coating composition, wherein the polymer of the first coating composition comprises a molecular weight and wherein the second coating composition comprises a polymer having a lower molecular weight than the molecular weight of the polymer of the first coating composition.

31. The method of claim 30, further comprising functionalizing at least part of the surface prior to applying the second coating composition.

32. A method of using the device of claim 14, wherein the method comprises implanting the device in an animal or a human body.

33. A vehicle for carrying a drug, wherein the vehicle comprises the coating composition of claim 1.
